SEQUENCE LISTING (9 sequences)

SEQ ID NO: 1 - protein, 538 aa, Artificial Sequence
  Synthetic
MSKGIKNSQLKKKNVKASNVAEKIEEKVEKTDKVVEKAAEVTEKRIRNLKLQEKVVTADVAADMIENGMIVAISGFTPSGYPKEVPKALTKKVNALEEEFKVTLYTGSSTGADIDGEWAKAGIIERRIPYQTNSDMRKKINDGSIKYADMHLSHMAQYINYSVIPKVDIAIIEAVAITEEGDIIPSTGIGNTATFVENADKVIVEINEAQPLELEGMADIYTLKNPPRREPIPIVNAGNRIGTTYVTCGSEKICAIVMTNTQDKTRPLTEVSPVSQAISDNLIGFLNKEVEEGKLPKNLLPIQSGVGSVANAVLAGLCESNFKNLSCYTEVIQDSMLKLIKCGKADVVSGTSISPSPEMLPEFIKDINFFREKIVLRPQEISNNPEIARRIGVISINTALEVDIYGNVNSTHVMGSKMMNGIGGSGDFARNAYLTIFTTESIAKKGDISSIVPMVSHVDHTEHDVMVIVTEQGVADLRGLSPREKAVAIIENCVHPDYKDMLMEYFEEACKSSGGNTPHNLEKALSWHTKFIKTGSMK

SEQ ID NO: 2 - protein, 451 aa, Artificial Sequence
  Synthetic
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 3 - protein, 371 aa, Artificial Sequence
  Synthetic
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 4 - protein, 431 aa, Artificial Sequence
  Synthetic
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 5 - protein, 388 aa, Artificial Sequence
  Synthetic
MMRFTLPRDIYYGKGSLEQLKNLKGKKAMLVLGGGSMKRFGFVDKVLGYLKEAGIEVKLIEGVEPDPSVETVFKGAELMRQFEPDWIIAMGGGSPIDAAKAMWIFYEHPEKTFDDIKDPFTVPELRNKAKFLAIPSTSGTATEVTAFSVITDYKTEIKYPLADFNITPDVAVVDSELAETMPPKLTAHTGMDALTHAIEAYVATLHSPFTDPLAMQAIEMINEHLFKSYEGDKEAREQMHYAQCLAGMAFSNALLGICHSMAHKTGAVFHIPHGCANAIYLPYVIKFNSKTSLERYAKIAKQISLAGNTNEELVDSLINLVKELNKKMQIPTTLKEYGIHEQEFKNKVDLISERAIGDACTGSNPRQLNKDEMKKIFECVYYGTEVDF

SEQ ID NO: 6 - protein, 431 aa, Artificial Sequence; identical to SEQ ID NO: 4
  Synthetic

SEQ ID NO: 7 - protein, 378 aa, Artificial Sequence
  Synthetic
YYGKGSLEQLKNLKGKKAMLVLGGGSMKRFGFVDKVLGYLKEAGIEVKLIEGVEPDPSVETVFKGAELMRQFEPDWIIAMGGGSPIDAAKAMWIFYEHPEKTFDDIKDPFTVPELRNKAKFLAIPSTSGTATEVTAFSVITDYKTEIKYPLADFNITPDVAVVDSELAETMPPKLTAHTGMDALTHAIEAYVATLHSPFTDPLAMQAIEMINEHLFKSYEGDKEAREQMHYAQCLAGMAFSNALLGICHSMAHKTGAVFHIPHGCANAIYLPYVIKFNSKTSLERYAKIAKQISLAGNTNEELVDSLINLVKELNKKMQIPTTLKEYGIHEQEFKNKVDLISERAIGDACTGSNPRQLNKDEMKKIFECVYYGTEVDF

SEQ ID NO: 8 - protein, 6 aa, Artificial Sequence
  Synthetic
GTCGAC

SEQ ID NO: 9 - protein, 6 aa, Artificial Sequence
  Synthetic
GAGCTC

(19) United States
(12) Patent Application Publication
(10) Pub. No.: US 2013/0217086 A1
(43) Pub. Date: Aug. 22, 2013

(54) MODIFIED MICROORGANISM FOR PRODUCTION OF 1,4-BUTANEDIOL

(71) Applicant: Samsung Electronics Co., Ltd., (US)

(72) Inventors: Young Min LEE, Suwon-si (KR); Hyun Min KOO, Seoul (KR); Jae Young KIM, Suwon-si (KR); Jin Woo KIM, Seoul (KR); Jae Chan PARK, Yongin-si (KR); Hwa Young CHO, Hwaseong-si (KR); Joon Song PARK, Seoul (KR); Pyung Cheon LEE, Suwon-si (KR)

(73) Assignee: SAMSUNG ELECTRONICS CO., LTD., Suwon-si (KR)

(21) Appl. No.: 13/725,256

(22) Filed: Dec. 21, 2012

(30) Foreign Application Priority Data
Feb. 15, 2012 (KR) 10-2012-0015526

(51) Int. Cl. C12P 7/18

(52) U.S. Cl. CPC C12P 7/18
USPC 435/135; 435/252.33; 435/252.32; 435/320.1; 435/158

(57) ABSTRACT

A modified microorganism for production of 1,4-butanediol, an expression vector, and a method of producing 1,4-butanediol using the modified microorganism are provided. The method can be useful in producing 1,4-butanediol using a biological production process.

### CROSS-REFERENCE TO RELATED APPLICATION

This application claims priority to Korean Patent Application No. 10-2012-0015526, filed on Feb. 2, 2012, and all the benefits accruing therefrom under 35 U.S.C. §119, the content of which in its entirety is herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 26,721 Byte ASCII (Text) file named “710930_ST25.TXT,” created on Dec. 20, 2012.

### BACKGROUND

1,4-Butanediol has been produced at a scale of approximately 1,000,000 tons or more all over the world, and used for various applications such as production of γ-butyrolactone (GBL), tetrahydrofuran (THF), pyrrolidone, N-methylpyrrolidone (NMP), etc.

In recent years, 1,4-butanediol has been produced by reaction of acetylene with two molecules of formaldehyde, followed by hydrogenation, and also produced by esterification and hydrogenation of maleic anhydride derived from butane. However, when 1,4-butanediol is produced using a chemical production process as described above, the production costs are increased due to an increase in oil price. Thus, development of a process capable of complementing and substituting the chemical production process is required.

### SUMMARY

A modified microorganism including a biosynthetic pathway for producing 1,4-butanediol is provided.

In an aspect, a modified microorganism for producing 1,4-butanediol, which converts α-ketoglutarate or succinate into 4-hydroxybutyryl-CoA, and converts the 4-hydroxybutyryl-CoA into 1,4-butanediol, is provided. The modified microorganism includes at least one heterologous polynucleotide selected from the group consisting of adh1, yiaY, adh4, adhB, mdh, eutG, fucO, dhaT, aldA, eutE, adhE1, adhE2 and adh2.

In another aspect, an expression vector is provided. The expression vector includes a polynucleotide including a nucleotide sequence for expressing an enzyme that converts α-ketoglutarate or succinate into 4-hydroxybutyryl-CoA, and a polynucleotide including a nucleotide sequence for expressing an enzyme that converts the 4-hydroxybutyryl-CoA into 1,4-butanediol, wherein the polynucleotide comprising a nucleotide sequence for expressing an enzyme that converts the 4-hydroxybutyryl-CoA into 1,4-butanediol is at least one selected from the group consisting of adh1, yiaY, adh4, adhB, mdh, eutG, fucO, dhaT, aldA, eutE, adhE1, adhE2 and adh2.

In still another aspect, a method of producing 1,4-butanediol is provided. The method includes culturing a modified microorganism in a glucose-containing medium, and recovering the 1,4-butanediol from the medium.

According to the method, the 1,4-butanediol may be produced using the biological production process.

### BRIEF DESCRIPTION OF THE DRAWINGS

The above and other aspects of this disclosure will become more readily apparent by describing in further detail non-limiting exemplary embodiments thereof with reference to the accompanying drawings, in which:

FIG. 1 shows a biosynthetic pathway of 1,4-butanediol.

FIG. 2 shows the results of measurement of a level of 1,4-butanediol produced in a modified microorganism prepared according to one exemplary embodiment.

### DETAILED DESCRIPTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art and are described in numerous standard texts and reference works.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a,” “an,” and “the” include the plural reference unless the context clearly indicates otherwise. Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

A modified microorganism for production of 1,4-butanediol using a biosynthetic pathway is provided.

The terms “biosynthetic pathway” and “metabolic pathway” used interchangeably in this specification refer to a series of at least two enzymatic reactions which take place in a host cell and one enzymatic reaction product becomes a substrate for performing the next chemical reaction. In each step of the metabolic pathway, an intermediate compound is formed, and then used as a substrate for the next step. These compounds are referred to as “metabolic intermediates,” and products obtained from the respective steps are referred to as “metabolites.”

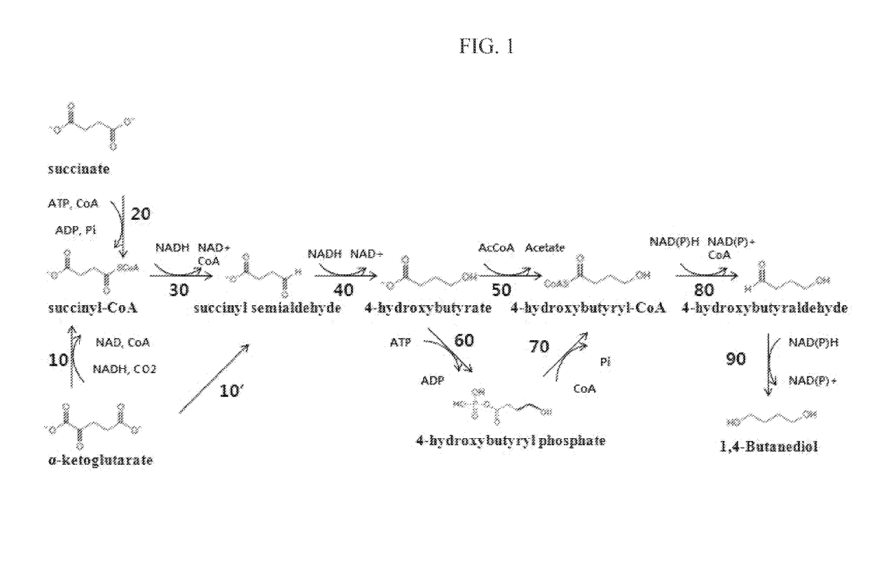

The term “1,4-butanediol” used in this specification refers to an organic compound which is represented by the formula C₄H₁₀O₂ (hereinafter, referred to as “1,4-butanediol”) and may be produced through two steps. The biosynthetic pathway of 1,4-butanediol is shown in FIG. 1.

In Step 1, α-ketoglutarate or succinate is converted into 4-hydroxybutyryl-CoA. More particularly, the α-ketoglutarate or succinate may be converted into 4-hydroxybutyryl-CoA via succinyl-CoA, succinyl semialdehyde and 4-hydroxybutyrate.

The α-ketoglutarate may be converted into succinyl-CoA by means of α-ketoglutarate dehydrogenase (10), and the succinate may be converted into succinyl-CoA by means of succinyl-CoA transferase (20). The succinyl-CoA is converted into succinyl semialdehyde by means of succinate semialdehyde dehydrogenase (30). Meanwhile, the α-ketoglutarate may be directly converted into succinyl semialdehyde by means of α-ketoglutarate decarboxylase (10′) without production of succinyl-CoA.

The succinyl semialdehyde is converted into 4-hydroxybutyrate by means of 4-hydroxybutanoate dehydrogenase (40). The 4-hydroxybutyrate may be converted into 4-hydroxybutyryl-CoA by means of 4-hydroxybutyryl-CoA transferase (50). Also, the 4-hydroxybutyrate may be converted into 4-hydroxybutyryl phosphate by means of butyrate kinase (60), and the 4-hydroxybutyryl phosphate may be converted into 4-hydroxybutyryl-CoA by means of phosphotransbutyrylase (70).

In Step 2, the 4-hydroxybutyryl-CoA is converted into 1,4-butanediol via 4-hydroxybutyraldehyde.

The 4-hydroxybutyryl-CoA may be converted into 4-hydroxybutyraldehyde by means of aldehyde dehydrogenase (80), and the 4-hydroxybutyraldehyde may be finally converted into 1,4-butanediol by means of alcohol dehydrogenase (90).

In one exemplary embodiment, a modified microorganism including an activity of converting α-ketoglutarate or succinate into 4-hydroxybutyryl-CoA, and an activity of converting the 4-hydroxybutyryl-CoA into 1,4-butanediol is provided.

As used herein, the term “metabolically engineered” or “metabolic engineering” involves rational pathway design and assembly of biosynthetic genes, genes associated with operons, and control elements of such nucleic acid sequences, for the production of a desired metabolite, such as an alcohol, in a microorganism. “Metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, or association with a heterologous expression control sequence in an endogenous host cell. Appropriate culture conditions are conditions such as culture medium pH, ionic strength, nutritive content, temperature, oxygen, CO₂, nitrogen content, humidity, and other culture conditions that permit production of the compound by the host microorganism, i.e., by the metabolic action of the microorganism. Appropriate culture conditions are well known for microorganisms that can serve as host cells.

Accordingly, a metabolically “engineered” or “modified” microorganism, which can also be called a “recombinant” microorganism, is produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite.

For example, the introduction of genetic material into a parental microorganism results in a new or modified ability to produce a chemical. The genetic material introduced into the parental microorganism contains one or more genes, or parts of genes, coding for one or more of the enzymes involved in a biosynthetic pathway for the production of a chemical and may also include additional elements for the expression or regulation of expression of these genes, e.g. promoter sequences.

In one exemplary embodiment, the microorganism may be modified to have an activity of converting α-ketoglutarate or succinate into 4-hydroxybutyryl-CoA, and an activity of converting the 4-hydroxybutyryl-CoA into 1,4-butanediol.

As used interchangeably herein, the terms “activity” and “enzymatic activity” refer to any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof.

The activity of converting α-ketoglutarate or succinate into 4-hydroxybutyryl-CoA may be exerted by at least one enzyme selected from the group consisting of α-ketoglutarate dehydrogenase, α-ketoglutarate decarboxylase, succinyl-CoA transferase, succinate semialdehyde dehydrogenase, 4-hydroxybutyrate dehydrogenase, 4-hydroxybutyryl-CoA transferase, butyrate kinase, and phosphotransbutyrylase. In one embodiment, the succinyl-CoA transferase, the succinate semialdehyde dehydrogenase, the 4-hydroxybutyrate dehydrogenase and the 4-hydroxybutyryl-CoA transferase may be used herein.

In one exemplary embodiment, the activity of converting α-ketoglutarate or succinate into 4-hydroxybutyryl-CoA may be exogenous.

As used herein, the term “exogenous” means that a genetic material of interest is not natural in a host strain (i.e., heterologous). The term “native” means that a genetic material is found in a genome of a wild-type cell in the host strain.

As used herein, the term “derived from” means that a genetic material is wholly or partially isolated from its given source or purified from the given source.

The activity of converting α-ketoglutarate or succinate into 4-hydroxybutyryl-CoA may be derived from all of prokaryotic and eukaryotic organisms such as archaebacteria, eubacteria, yeasts, plants, insects, animals and humans. For example, the microorganism may be at least one selected from the group consisting of Escherichia coli, Saccharomyces cerevisiae, Clostridium kluyveri, Clostridium acetobutylicum, Clostridium beijerinckii, Clostridium saccharoperbutylacetonicum, Clostridium perfringens, Clostridium difficile, Ralstonia eutropha, Mycobacterium bovis, Mycobacterium tuberculosis, Porphyromonas gingivalis and Corynebacterium glutamicum, but the present invention is not limited thereto. In one embodiment, succinyl-CoA transferase (NCBI GenBank Aceession NO. P38946.1) derived from C. kluyveri, and succinate semialdehyde dehydrogenase (NCBI GenBank Aceession NO. YP_{—}004510528.1), 4-hydroxybutyrate dehydrogenase (NCBI GenBank Aceession NO. YP_{—}004510529.1) and 4-hydroxybutyryl CoA transferase (NCBI GenBank Aceession NO. EIW94739.1) derived from P. gingivalis are used herein.

The activity of converting the 4-hydroxybutyryl-CoA into 1,4-butanediol may be exerted by at least one enzyme selected from the group consisting of aldehyde dehydrogenase (NCBI GenBank Aceession NO. CAA78962.1) and alcohol dehydrogenase (NCBI GenBank Aceession NO. CAA44614.1). In one embodiment, the aldehyde dehydrogenase may be used herein.

In one exemplary embodiment, the activity of converting the 4-hydroxybutyryl-CoA into the 1,4-butanediol may be exogenous, and may be derived from any prokaryotic or eukaryotic organisms, such as archaebacteria, eubacteria, yeasts, plants, insects, animals and humans. For example, the microorganism may be selected from the group consisting of Clostridium saccharobutylicum, E. coli, Schizosaccharomyces pombe, Zymomonas mobilis, Bacillus methanolicus, Klebsiella pneumonia, Salmonella typhimurium, Clostridium ljungdahlii, Clostridium butyricum, Entamoeba histolytica and C. glutamicum, but the present invention is not limited thereto. In one embodiment, aldehyde dehydrogenase (NCBI GenBank Aceession NO. CAQ57983) derived from C. saccharobutylicum may be used herein.

Conventional methods known in the art may be used to introduce the activity of converting α-ketoglutarate or succinate into 4-hydroxybutyryl-CoA and the activity of converting the 4-hydroxybutyryl-CoA into 1,4-butanediol into a microorganism. For example, a method that includes constructing an expression vector including a polynucleotide for expressing the activities (e.g., enzymes) and transforming the expression vector into a microorganism may be used herein.

In another exemplary embodiment, an expression vector that includes a polynucleotide including a nucleotide sequence for expressing (e.g., encoding) an enzyme that converts α-ketoglutarate or succinate into 4-hydroxybutyryl-CoA, and a polynucleotide including a nucleotide sequence for expressing an enzyme that converts the 4-hydroxybutyryl-CoA into 1,4-butanediol is provided. In one exemplary embodiment, the polynucleotide including a nucleotide sequence for expressing an enzyme that converts the 4-hydroxybutyryl-CoA into 1,4-butanediol may be at least one selected from the group consisting of adh1, yiaY, adh4, adhB, mdh, eutG, fucO, dhaT, aldA, eutE, adhE1, adhE2 and adh2. Other polynucleotides can be used that encode the same or functionally equivalent gene products.

As used herein, the term “expression vector” refers to a DNA construct containing a DNA sequence that is operably linked to a suitable control sequence capable of effecting the expression of the DNA in a suitable host. The vector may be a plasmid, a phage particle, or simply a potential genomic insert. Once transformed into a suitable host, the vector replicates and functions independently of the host genome, or integrates into the genome itself. As used herein, the terms “plasmid,” “expression plasmid,” and “vector” are often used interchangeably as a plasmid is among the most commonly used forms of vector at present.

However, it is intended to include such other forms of expression vectors that serve equivalent functions and which are, or become, known in the art. For example, the vector may be a cloning vector, an expression vector, a shuttle vector, a plasmid, a phage or virus particle, a DNA construct, or a cassette. As used herein, the term “plasmid” refers to a circular double-stranded DNA construct used as a cloning vector, and which forms an extra chromosomal self-replicating genetic element in many bacteria and some eukaryotes. The plasmid may be a multicopy plasmid that can integrate into the genome of the host cell by homologous recombination.

As known to those skilled in the art, in order to increase the expression level of a gene introduced to a host cell, the gene should be operably linked to expression control sequences for the control of transcription and translation which function in the selected expression host. For example, the expression control sequences and the gene are included in one expression vector together with a selection marker and a replication origin. When the expression host is a eukaryotic cell, the expression vector should further include an expression marker useful in the eukaryotic expression host.

As used herein, the term “operably linked” indicates that elements are arranged to perform the general functions of the elements. A nucleic acid is said to be “operably linked” when it is placed into a functional relationship with another nucleic acid sequence. For example, a polynucleotide promoter sequence is operably linked to a polynucleotide encoding a polypeptide if it affects the transcription of the sequence. The term “operably linked” may mean that the polynucleotide sequences being linked are contiguous. Linking may be accomplished by ligation at convenient restriction sites. If such sites do not exist, synthetic oligonucleotide adaptors or linkers may be used in accordance with conventional practice.

As used herein, the term “promoter” refers to a nucleic acid sequence that functions to drive or effect transcription of a downstream gene. The promoter may be any promoter that drives expression of a target protein, and may be any nucleic acid sequence which shows transcriptional activity in the host cell of choice. Also, the promoter includes mutant, truncated and hybrid promoters, and may be obtained from genes encoding extracellular or intracellular polypeptides either homologous or heterologous to the host cell. The promoter sequence may be native or foreign to the host cell.

As used herein, the term “gene” refers to a nucleotide sequence that encodes a gene product, such as a protein or enzyme, including a chromosomal or non-chromosomal segment of DNA involved in producing a polypeptide chain that may or may not include regions preceding and following the coding regions, for example, 5′ untranslated (“5′ UTR”) or leader sequences and 3′ untranslated (“3′ UTR”) or trailer sequences, as well as intervening sequence (introns) between individual coding segments (exons).

As used interchangeably herein, the terms “polynucleotide” and “nucleic acid” refer to a polymeric form of nucleotides of any length. These terms may include, but are not limited to, a single-stranded DNA (“deoxyribonucleic acid”), double-stranded DNA, genomic DNA, cDNA, or a polymer comprising purine and pyrimidine bases, or other natural, chemically-modified, biochemically-modified, non-natural or derivatized nucleotide bases. Non-limiting examples of polynucleotides include genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA (“ribonucleic acid”) of any sequence, nucleic acid probes, and primers. It will be understood that, as a result of the degeneracy of the genetic code, a multitude of nucleotide sequences encoding a given protein may be produced.

In the embodiment, the promoter may be, but is not limited to, selected from the group consisting of GAP (“glyceraldehyde-3-phosphate dehydrogenase”), PGK1 (“phosphoglycerate kinase 1”), CYC (“cytochrome-c oxidase”), TEF (“translation elongation factor 1α”), ADH (“alcohol dehydrogenase”), PHO5, TRP1, GAL1, GAL10, hexokinase, pyruvate decarboxylase, phosphofructokinase, triose phosphate isomerase, phosphoglucose isomerase, glucokinase, α-mating factor pheromone, GUT2, nmt, fbp1, AOX1, AOX2, MOX1 and, FMD1. In an exemplary embodiment, GPD promoter is used.

In one exemplary embodiment, the polynucleotide encoding an enzyme that converts α-ketoglutarate or succinate into 4-hydroxybutyryl-CoA may be at least one selected from the group consisting of a polynucleotide including a nucleotide sequence for expressing α-ketoglutarate dehydrogenase, a polynucleotide including a nucleotide sequence for expressing α-ketoglutarate decarboxylase, a polynucleotide including a nucleotide sequence for expressing succinyl-CoA transferase, a polynucleotide including a nucleotide sequence for expressing succinate semialdehyde dehydrogenase, a polynucleotide including a nucleotide sequence for expressing 4-hydroxybutyrate dehydrogenase, a polynucleotide including a nucleotide sequence for expressing 4-hydroxybutyryl-CoA transferase, a polynucleotide including a nucleotide sequence for expressing g butyrate kinase, and a polynucleotide including a nucleotide sequence for expressing phosphotransbutyrylase. In one embodiment, the polynucleotide including a nucleotide sequence for expressing succinyl-CoA transferase, the polynucleotide including a nucleotide sequence for expressing succinate semialdehyde dehydrogenase, the polynucleotide including a nucleotide sequence for expressing 4-hydroxybutyrate dehydrogenase and the polynucleotide including a nucleotide sequence for expressing 4-hydroxybutyryl CoA transferase may be used herein.

The succinyl-CoA transferase can be encoded by a nucleic acid including any nucleotide sequence, such as a cat1 gene. The cat1 gene may include a nucleotide sequence encoding the amino acid sequence of SEQ ID NO: 1, or an amino acid sequence comprising at least about 70%, at least about 75%, at least about 80%, at least about 85%, at least about 90%, at least about 92%, at least about 95%, at least about 97%, at least about 98% or at least about 99% sequence homology (e.g., sequence identity) to the amino acid sequence of SEQ ID NO: 1.

SEQ ID NO: 1 (538 aa):

| | |
|---|---|
| MSKGIKNSQLKKKNVKASNVAEKIEEKVEKTDKVVEKAAEVTEKRI | 50 |
| RNLK | |
| LQEKVVTADVAADMIENGMIVAISGFTPSGYPKEVPKALTKKVNAL | 100 |
| EEEF | |
| KVTLYTGSSTGADIDGEWAKAGIIERRIPYQTNSDMRKKINDGSIK | 150 |
| YADM | |
| HLSHMAQYINYSVIPKVDIAIIEAVAITEEGDIIPSTGIGNTATFV | 200 |
| ENAD | |
| KVIVEINEAQPLELEGMADIYTLKNPPRREPIPIVNAGNRIGTTYV | 250 |
| TCGS | |
| EKICAIVMTNTQDKTRPLTEVSPVSQAISDNLIGFLNKEVEEGKLP | 300 |
| KNLL | |
| PIQSGVGSVANAVLAGLCESNFKNLSCYTEVIQDSMLKLIKCGKAD | 350 |
| VVSG | |
| TSISPSPEMLPEFIKDINFFREKIVLRPQEISNNPEIARRIGVISI | 400 |
| NTAL | |
| EVDIYGNVNSTHVMGSKMMNGIGGSGDFARNAYLTIFTTESIAKKG | 450 |
| DISS | |
| IVPMVSHVDHTEHDVMVIVTEQGVADLRGLSPREKAVAIIENCVHP | 500 |
| DYKDMLMEYFEEACKSSGGNTPHNLEKALSWHTKFIKTGSMK | |

The succinate semialdehyde dehydrogenase can be encoded by a nucleic acid including any nucleotide sequence such as a SucD gene. The SucD gene may include a nucleotide sequence encoding the amino acid sequence of SEQ ID NO: 2, or an amino acid sequence comprising at least about 70%, at least about 75%, at least about 80%, at least about 85%, at least about 90%, at least about 92%, at least about 95%, at least about 97%, at least about 98% or at least about 99% sequence homology to the amino acid sequence of SEQ ID NO: 2.

SEQ ID NO: 2 (451 aa):

| | |
|---|---|
| MEIKEMVSLARKAQKEYQATHNQEAVDNICRAAAKVIYENAAILAR | 50 |
| EAVD | |
| ETGMGVYEHKVAKNQGKSKGVWYNLHNKKSIGILNIDERTGMIEIA | 100 |
| KPIG | |
| VVGAVTPTTNPIVTPMSNIIFALKTCNAIIIAPHPRSKKCSAHAVR | 150 |
| LIKE | |
| AIAPFNVPEGMVQIIEEPSIEKTQELMGAVDVVVATGGMGMVKSAY | 200 |
| SSGK | |
| PSFGVGAGNVQVIVDSNIDFEAAAEKIITGRAFDNGIICSGEQSII | 250 |
| YNEA | |
| DKEAVFTAFRNHGAYFCDEAEGDRARAAIFENGAIAKDVVGQSVAF | 300 |
| IAKK | |
| ANINIPEGTRILVVEARGVGAEDVICKEKMCPVMCALSYKHFEEGV | 350 |
| EIAR | |
| TNLANEGNGHTCAIHSNNQAHIILAGSELTVSRIVVNAPSATTAGG | 400 |
| HIQN | |
| GLAVTNTLGCGSWGNNSISENFTYKHLLNISRIAPLNSSIHIPDDK | 450 |
| EIWEL | |

The 4-hydroxybutyrate dehydrogenase can be encoded by a nucleic acid including any nucleotide sequence such as a 4hbd gene. The 4hbd gene may include a nucleotide sequence encoding the amino acid a sequence of SEQ ID NO: 3, or an amino acid sequence comprising at least about 70%, at least about 75%, at least about 80%, at least about 85%, at least about 90%, at least about 92%, at least about 95%, at least about 97%, at least about 98% or at least about 99% sequence homology to the amino acid sequence of SEQ ID NO: 3.

SEQ ID NO: 3 (371 aa):

| | |
|---|---|
| MQLFKLKSVTHHFDTFAEFAKEFCLGERDLVITNEFIYEPYMKACQ | 50 |
| LPCH | |
| FVMQEKYGQGEPSDEMMNNILADIRNIQFDRVIGIGGGTVIDISKL | 100 |
| FVLK | |
| GLNDVLDAFDRKIPLIKEKELIIVPTTCGTGSEVTNISIAEIKSRH | 150 |
| TKMG | |
| LADDAIVADHAIIIPELLKSLPFHFYACSAIDALIHAIESYVSPKA | 200 |
| SPYS | |
| RLFSEAAWDIILEVFKKIAEHGPEYRFEKLGEMIMASNYAGIAFGN | 250 |
| AGVG | |
| AVHALSYPLGGNYHVPHGEANYQFFTEVFKVYQKKNPFGYIVELNW | 300 |
| KLSK | |
| ILNCQPEYVYPKLDELLGCLLTKKPLHEYGMKDEEVRGFAESVLKT | 350 |

QQRLLANNYVELTVDEIEGIYRRLY

The 4-hydroxybutyryl CoA transferase can be encoded by a nucleic acid including any nucleotide sequence such as a ghb gene. The ghb gene may include a nucleotide sequence encoding the amino acid sequence of SEQ ID NO: 4, or an amino acid sequence comprising at least about 70%, at least about 75%, at least about 80%, at least about 85%, at least about 90%, at least about 92%, at least about 95%, at least about 97%, at least about 98% or at least about 99% sequence homology to the amino acid sequence of SEQ ID NO: 4.

SEQ ID NO: 4 (431 aa):

| | |
|---|---|
| MKDVLAEYASRIVSAEEAVKHIKNGERVALSHAAGVPQSCVDALVQ | 50 |
| QADL | |
| FQNVEIYHMLCLGEGKYMAPEMAPHFRHITNFVGGNSRKAVEENRA | 100 |
| DFIP | |
| VFFYEVPSMIRKDILHIDVAIVQLSMPDENGYCSFGVSCDYSKPAA | 150 |
| ESAH | |
| LVIGEINRQMPYVHGDNLIHISKLDYIVMADYPIYSLAKPKIGEVE | 200 |
| EAIG | |
| RNCAELIEDGATLQLGIGAIPDAALLFLKDKKDLGIHTEMFSDGVV | 250 |
| ELVR | |
| SGVITGKKKTLHPGKMVATFLMGSEDVYHFIDKNPDVELYPVDYVN | 300 |
| DPRV | |
| IAQNDNMVSINSCIEIDLMGQVVSECIGSKQFSGTGGQVDYVRGAA | 350 |
| WSKN | |
| GKSIMAIPSTAKNGTASRIVPIIAEGAAVTTLRNEVDYVVTEYGIA | 400 |
| QLKGKSLRQRAEALIAIAHPDFREELTKHLRKRFG | |

In the embodiment, a polynucleotide encoding an enzyme that converts the 4-hydroxybutyryl-CoA into 1,4-butanediol may be at least one selected from the group consisting of a polynucleotide encoding aldehyde dehydrogenase and a polynucleotide encoding alcohol dehydrogenase.

For example, a polynucleotide encoding an enzyme that converts the 4-hydroxybutyryl-CoA into the 1,4-butanediol may be at least one selected from the group consisting of adh1, yiaY, adh4, adhB, mdh, eutG, fucO, dhaT, aldA, eutE, adhE1, adhE2 and adh2.

In one exemplary embodiment, a polynucleotide encoding aldehyde dehydrogenase was used herein.

For example, the aldehyde dehydrogenase can be encoded by a nucleic acid including any nucleotide sequence such as a adh1 gene. The adh1 gene may include a nucleotide sequence encoding the amino acid sequence of SEQ ID NO: 5, or an amino acid sequence comprising at least about 70%, at least about 75%, at least about 80%, at least about 85%, at least about 90%, at least about 92%, at least about 95%, at least about 97%, at least about 98% or at least about 99% sequence homology to the amino acid sequence of SEQ ID NO: 5.

SEQ ID NO: 5 (388 aa):
MMRFTLPRDIYYGKGSLEQLKNLKGKKAMLVLGGGSMKRFGFVDKVLGYL
KEAGIEVKLIEGVEPDPSVETVFKGAELMRQFEPDWIIAMGGGSPIDAAKAMWIFYEHPE
KTFDDIKDPFTVPELRNKAKFLAIPSTSGTATEVTAFSVITDYKTEIKYPLADFNITPDVAV
VDSELAETMPPKLTAHTGMDALTHAIEAYVATLHSPFTDPLAMQAIEMINEHLFKSYEG
DKEAREQMHYAQCLAGMAFSNALLGICHSMAHKTGAVFHIPHGCANAIYLPYVIKFNS
KTSLERYAKIAKQISLAGNTNEELVDSLINLVKELNKKMQIPTTLKEYGIHEQEFKNKVD
LISERAIGDACTGSNPRQLNKDEMKKIFECVYYGTEVDF

As used herein, the term “homology” refers to sequence similarity or sequence identity. This homology or identity (e.g., percent identity) may be determined using standard techniques known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

The expression vector may be introduced to a host cell by a known method in the art.

As used herein, the term “host cell” refers to a suitable cell that serves as a host for an expression vector. A suitable host cell may be a naturally occurring or wild-type host cell, or it may be an altered host cell. A “wild-type host cell” is a host cell that has not been genetically altered using recombinant methods.

As used herein, the term “altered host cell” refers to a genetically engineered host cell wherein a gene is expressed at an altered level of expression compared to the level of expression of the same gene in an unaltered or wild-type host cell grown under the same growth conditions. A “modified host cell” herein refer to a wild-type or altered host cell that has been genetically engineered to express or overexpress a non-native or other target protein. A modified host cell is preferably capable of producing 1,4-butanediol at a greater level than its wild-type or altered parent host cell.

In the embodiment, the host cell may be, but is not limited to, selected from the group consisting of Escherichia, Klebsiella, Bacillus, Corynebacterium, Zymomonas, Lactococcus, Lactobacillus, Streptomyces, Clostridium, Pseudomonas, Alcaligenes, Salmonella, Shigella, Burkholderia, Aspergillus, Oligotropha, Pichia, Candida, Hansenula, Saccharomyces, and Kluyveromyces. In an exemplary embodiment, a Corynebacterium glutamicum was used.

As used herein, the term “introduced” refers to any method suitable for transferring the nucleic acid sequence into the cell, such that it can be expressed. Such a method for introduction may be, but is not limited to, protoplast fusion, transfection, transformation, conjugation, and transduction (See e.g., Ferrari et al., “Genetics,” in Hardwood et al., (eds.), Bacillus, Plenum Publishing Corp., pages 57-72, [1989]).

As used herein, the terms “transformed” and “stably transformed” refer to a cell that has a non-native heterologous polynucleotide sequence integrated into its genome or has the heterologous polynucleotide sequence present as an episomal plasmid that is maintained for at least two generations.

The introduction of the polynucleotide encoding a polypeptide having the activity of converting α-ketoglutarate or succinate into 4-hydroxybutyryl-CoA, and the polynucleotide encoding a polypeptide having an activity of converting the 4-hydroxybutyryl-CoA into 1,4-butanediol to a host cell may be performed by isolating a plasmid from E. coli and then by transforming the plasmid into the host cell. However, it is not essential to use intervening microorganisms such as E. coli. A vector comprising polynucleotides encoding enzymes with the desired activity can be directly introduced into a host cell. Transformation may be achieved by any one of various means including electroporation, microinjection, biolistics (or particle bombardment-mediated delivery), or agrobacterium-mediated transformation.

In one embodiment, the host cell is of a strain deposited on Feb. 13, 2012 with the Korea Research Institute of Bioscience and Biotechnology under accession number KCTC 12137BP.

In another embodiment, a method of producing 1,4-butanediol using the modified microorganism is provided. The method may include culturing the modified microorganism in a glucose-containing medium, and recovering 1,4-butanediol from the medium.

The medium used to culture the cells may include any conventional suitable medium known in the art for growing the host cells, such as minimal or complex media containing appropriate supplements. Suitable media are available from commercial suppliers or may be prepared according to published recipes (e.g., in catalogues of the American Type Culture Collection).

In an exemplary embodiment, the medium may be a fermentation medium containing sugars that can be fermented by a genetically modified microorganism. The sugar may be a hexose, for example, glucose, glycan or another polymer of glucose, a glucose oligomer, for example, maltose, maltotriose or isomaltotriose, panose, fructose or a fructose oligomer. In addition, the fermentation medium may contain nitrogen sources such as ammonia, ammonium sulfate, ammonium chloride, ammonium nitrate and urea; inorganic salts such as potassium monohydrogen phosphate, potassium dihydrogen phosphate and magnesium sulfate; and optionally a nutrient including various vitamins such as peptone, a meat extract, a yeast extract, a corn steep liquor, casamino acid, biotin and thiamine.

The modified microorganism may be cultured under batch, fed-batch or continuous fermentation conditions. Classical batch fermentation methods use a closed system, wherein the culture medium is made prior to the beginning of the fermentation run, the medium is inoculated with the desired organisms, and fermentation occurs without the subsequent addition of any components to the medium. In certain cases, the pH and oxygen content of the growth medium, but not the carbon source content, are altered during batch methods. The metabolites and cell biomass of the batch system change constantly up to the time the fermentation is stopped. In a batch system, cells usually progress through a static lag phase to a high growth log phase and finally to a stationary phase where growth rate is diminished or halted. If untreated, cells in the stationary phase eventually die. Generally, cells produce the most protein in the log phase.

A variation on the standard batch fermentation is a “fed-batch fermentation” system. In fed-batch fermentation, nutrients (e.g., a carbon source, nitrogen source, O₂, and typically, other nutrients) are only added when their concentration in culture falls below a threshold. Fed-batch systems are useful when catabolite repression is apt to inhibit the metabolism of the cells and where it is desirable to have limited amounts of nutrients in the medium. Actual nutrient concentration in fed-batch systems are estimated on the basis of the changes of measurable factors such as pH, dissolved oxygen and the partial pressure of waste gases such as CO₂. Batch and fed-batch fermentations are common and well known in the art.

Continuous fermentation employs an open system in which a defined culture medium is added continuously to a bioreactor and an equal amount of conditioned medium is removed simultaneously for processing. Continuous fermentation generally maintains the cultures at a constant high density where cells are primarily in log-phase growth. Continuous fermentation allows for the modulation of one factor or any number of factors that affect cell growth and/or end product concentration. For example, a limiting nutrient such as the carbon source or nitrogen source is maintained at a fixed rate and all other parameters are allowed to moderate. In other systems, a number of factors affecting growth are altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are known to those of skill in the art.

The step of recovering the chemical from the medium may be performed by any suitable method. For example, the method may include salting-out, recrystallization, extraction with organic solvent, esterification distillation, chromatography, and electrodialysis, and the method for separation, purification, or collection may be appropriately selected according to the characteristics of the chemical.

Alternatively, the method may further include forming polybutylene succinate (PBS) from the recovered 1,4-butanediol. For example, PBS may be produced by condensation polymerization of the recovered 1,4-butanediol with succinic acid or dimethyl-succinate. The PBS is an aliphatic polyester-based polymer which has excellent biodegradability and formability. Therefore, the PBS may be used for fishing nets, films and packaging vessels.

Alternatively, the method may further include forming polybutylene terephthalate (PBT) from the recovered 1,4-butanediol. For example, PBT may be produced by condensation polymerization of the recovered 1,4-butanediol with terephthalic acid or dimethyl-terephthalate. The PBT is a polyester-based polymer which has excellent crystallinity, dimensional stability and formability. Therefore, the PBT may be used for electrical and electronics and automotive parts, and also used as an engineering plastic material.

Hereinafter, the invention will be described in further detail with respect to exemplary embodiments. However, it should be understood that the invention is not limited to these Examples and may be embodied in various modifications and changes.

### EXAMPLES

### Strain and Plasmid

E. coli TOP10 F-mcrA Δ (mrr-hsdRMS-mcrBC) φ80lacZΔM15 ΔlacX74 nupG recA1 araD139 Δ (ara-leu) 7697 galE15 galK16 rpsL (StrR) endA1 λ-XL1 Blue (endA1 gyrA96 (nalR) thi-1 recA1 relA1 lac glnV44 F′[::Tn10 proAB+ lacIq ΔlacZ)M15] hsdR17 (rK− mK+) (Invitrogen, CA) was used as a host for proliferating and extracting a large amount of plasmid DNA. C. glutamicum ATCC13032 was used as a microbial host cell for producing 1,4-butanediol. A pET2 plasmid was used to express an episome plasmid. Also, a pK18mobsacB (ATCC 87097) plasmid for integration into the genome was used herein.

### Medium and Incubation

E. coli was seeded in an LB medium (1% bacto-trypton, 0.5% bacto-yeast extract, 1% NaCl) supplemented with kanamycin, and incubated at 37° C. The microbial host cell and the recombinant microorganism were incubated at a temperature of 30° C. in an LB-glucose medium (1% bacto-trypton, 0.5% bacto-yeast extract, 1% NaCl, 2% glucose). The transformed microbial host cell was incubated in a medium supplemented with kanamycin. All the strains were used herein.

### Example 1

The following example illustrates the construction of an expression vector in accordance with the invention.

A. Construction of pK18mobsacB-cat1-sucD-4hbd-cat2

Base sequences of a cat1 (NCBI GenBank Gene ID NO. 5392695) gene derived from C. kluyveri and a sucD (NCBI GenBank Gene ID NO. 10721855) gene, a 4hbd (NCBI GenBank Gene ID NO. 10722749) gene and a cat2 gene derived from P. gingivalis were modified (codon-optimized) and synthesized so as to express optimum levels of the genes in a microbial host cell, C. glutamicum.

The cat2 gene derived from P. gingivalis may include a nucleotide sequence encoding the amino acid sequence of SEQ ID NO: 6.

SEQ ID NO: 6 (431aa):

| | |
|---|---|
| MKDVLAEYASRIVSAEEAVKHIKNGERVALSHAAGVPQSCVDALVQ | 50 |
| QADL | |
| FQNVEIYHMLCLGEGKYMAPEMAPHFRHITNFVGGNSRKAVEENRA | 100 |
| DFIP | |
| VFFYEVPSMIRKDILHIDVAIVQLSMPDENGYCSFGVSCDYSKPAA | 150 |
| ESAH | |
| LVIGEINRQMPYVHGDNLIHISKLDYIVMADYPIYSLAKPKIGEVE | 200 |
| EAIG | |
| RNCAELIEDGATLQLGIGAIPDAALLFLKDKKDLGIHTEMFSDGVV | 250 |
| ELVR | |
| SGVITGKKKTLHPGKMVATFLMGSEDVYHFIDKNPDVELYPVDYVN | 300 |
| DPRV | |
| IAQNDNMVSINSCIEIDLMGQVVSECIGSKQFSGTGGQVDYVRGAA | 350 |
| WSKN | |
| GKSIMAIPSTAKNGTASRIVPIIAEGAAVTTLRNEVDYVVTEYGIA | 400 |
| QLKGKSLRQRAEALIAIAHPDFREELTKHLRKRFG | |

Information on a synthetic gene is as follows:

Base sequence (TCTAGA) of restriction enzyme XbaI-GAP promoter-cat1 gene-sucD gene-4hbd gene-cat2 gene-base sequence (GCTAGC) of restriction enzyme NheI

Next, the gene was digested with restriction enzymes XbaI and NheI, and introduced into pK18mobsacB, which had been digested with the same restriction enzymes, to construct pK18mobsacB-cat1-sucD-4hbd-cat2.

B. Construction of pET2-adh1

A base sequence of an adh1 gene derived from C. saccharobutylicum was modified and synthesized so as to express an optimum level of the adh1 gene in a microbial host cell, C. glutamicum.

The adh1 gene derived from C. glutamicum may include a nucleotide sequence encoding the amino acid sequence of SEQ ID NO: 7.

SEQ ID NO: 7 (378 aa):

| | |
|---|---|
| YYGKGSLEQLKNLKGKKAMLVLGGGSMKRFGFVDKVLGYLKEAGIE | 50 |
| VKLI | |
| EGVEPDPSVETVFKGAELMRQFEPDWIIAMGGGSPIDAAKAMWIFY | 100 |
| EHPE | |
| KTFDDIKDPFTVPELRNKAKFLAIPSTSGTATEVTAFSVITDYKTE | 150 |
| IKYP | |
| LADFNITPDVAVVDSELAETMPPKLTAHTGMDALTHAIEAYVATLH | 200 |
| SPFT | |
| DPLAMQAIEMINEHLFKSYEGDKEAREQMHYAQCLAGMAFSNALLG | 250 |
| ICHS | |
| MAHKTGAVFHIPHGCANAIYLPYVIKFNSKTSLERYAKIAKQISLA | 300 |
| GNTN | |
| EELVDSLINLVKELNKKMQIPTTLKEYGIHEQEFKNKVDLISERAI | 350 |
| GDACTGSNPRQLNKDEMKKIFECVYYGTEVDF | |

Information on a synthetic gene is as follows:

Base sequence (GTCGAC) of restriction enzyme SalI-GAP promoter-adhI gene-base sequence (GAGCTC) of restriction enzyme SacI

Next, the gene was digested with restriction enzymes SalI and SacI, and introduced into pET2, which had been digested with the same restriction enzymes, to construct pET2-adh1.

### Example 2

The following example illustrates the preparation of modified C. glutamicum.

The expression vector constructed in Example 1 was introduced into a C. glutamicum strain.

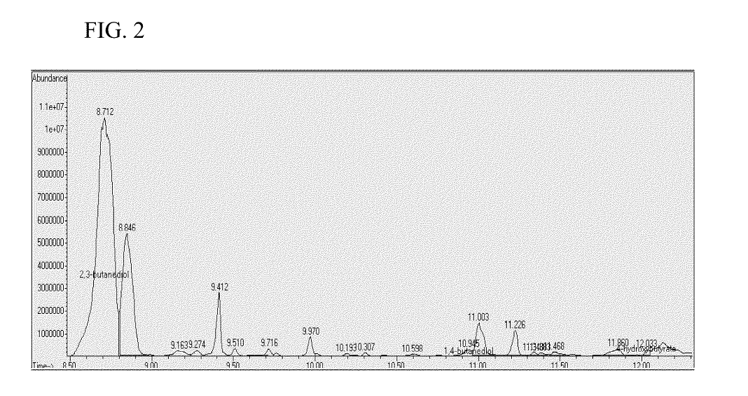

More particularly, the expression vector, pK18mobsacB-cat1-sucD-4hbd-cat2, constructed in Example 1-A was integrated into a cat1-sucD-4hbd-cat2 gene in the genome of C. glutamicum using an electroporation method. Next, the expression vector, pET2-adh1, constructed in Example 1-B was transformed into C. glutamicum using an electroporation method, and a level of 1,4-butanediol produced in the C. glutamicum was measured. The results are listed in Table 1 and shown in FIG. 2.

The C. glutamicum in which the cat1-sucD-4hbd-cat2 gene was integrated into the genome and into which the pET2-adh1 was introduced was deposited in the Korean Research Institute of Bioscience and Biotechnology on Feb. 13, 2012 under Accession No.: KCTC 12137BP.

TABLE 1

| Origin of LDH gene | 1,4-butanediol (mg/L) |
|---|---|
| adh1 | 24 |

As shown in FIG. 2, it could be confirmed that 1,4-butanediol was produced. More particularly, the 1,4-butanediol was produced at a level of 24 mg/L in the C. glutamicum strain including the adh1 gene.

Therefore, the C. glutamicum strains including the adh1 gene prepared in the Examples can produce the 1,4-butanediol. Thus, 1,4-butanediol, which has been widely used in chemical industries, can be produced using the biological production process described herein.

The description elaborates certain parts of the invention herein, and the description of specific details intends to proffer embodiments for one skilled in the art; therefore, the description does not limit the scope of the invention. As a result, the actual scope of the invention should be determined by the construction of the claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A modified microorganism for producing 1,4-butanediol, wherein the modified microorganism
converts α-ketoglutarate or succinate into 4-hydroxybutyryl-CoA;
converts the 4-hydroxybutyryl-CoA into 1,4-butanediol, and
comprises at least one heterologous polynucleotide sequence selected from the group consisting of adh1, yiaY, adh4, adhB, mdh, eutG, fucO, dhaT, aldA, eutE, adhE1, adhE2 adh2, and polynucleotide sequences encoding the gene products thereof.

2. The modified microorganism according to claim 1, wherein the microorganism converts α-ketoglutarate or succinate into 4-hydroxybutyryl-CoA using at least one enzyme selected from the group consisting of α-ketoglutarate dehydrogenase, α-ketoglutarate decarboxylase, succinyl-CoA transferase, succinate semialdehyde dehydrogenase, 4-hydroxybutyrate dehydrogenase, 4-hydroxybutyryl-CoA transferase, butyrate kinase, and phosphotransbutyrylase.

3. The modified microorganism according to claim 1, wherein the microorganism converts 4-hydroxybutyryl-CoA into 1,4-butanediol using at least one enzyme selected from the group consisting of aldehyde dehydrogenase and alcohol dehydrogenase.

4. The modified microorganism according to claim 2, wherein the microorganism converts α-ketoglutarate or succinate into 4-hydroxybutyryl-CoA using at least one enzyme derived from Escherichia coli, Saccharomyces cerevisiae, Clostridium kluyveri, Clostridium acetobutylicum, Clostridium beijerinckii, Clostridium saccharoperbutylacetonicum, Clostridium perfringens, Clostridium difficile, Ralstonia eutropha, Mycobacterium bovis, Mycobacterium tuberculosis, Porphyromonas gingivalis or Corynebacterium glutamicum.

5. The modified microorganism according to claim 3, wherein the microorganism converts 4-hydroxybutyryl-CoA into 1,4-butanediol using at least one enzyme derived from Clostridium saccharobutylicum, Escherichia coli, Schizosaccharomyces pombe, Zymomonas mobilis, Bacillus methanolicus, Klebsiella pneumonia, Salmonella typhimurium, Clostridium ljungdahlii, Clostridium butyricum, Entamoeba histolytica or Corynebacterium glutamicum.

6. The modified microorganism according to claim 1, wherein the modified microorganism is a modified Escherichia, Klebsiella, Bacillus, Corynebacterium, Zymomonas, Lactococcus, Lactobacillus, Streptomyces, Clostridium, Pseudomonas, Alcaligenes, Salmonella, Shigella, Burkholderia, Aspergillus, Oligotropha, Pichia, Candida, Hansenula, Saccharomyces or Kluyveromyces.

7. The modified microorganism according to claim 6, wherein the modified microorganism is a modified Escherichia coli.

8. The modified microorganism according to claim 6, wherein the modified microorganism is a modified Corynebacterium glutamicum.

9. The modified microorganism according to claim 1, wherein the modified microorganism is of a strain deposited under accession number KCTC 12137BP.

10. An expression vector comprising:
a polynucleotide comprising a nucleotide sequence encoding an enzyme that converts α-ketoglutarate or succinate into 4-hydroxybutyryl-CoA; and
a polynucleotide comprising a nucleotide sequence encoding an enzyme that converts 4-hydroxybutyryl-CoA into 1,4-butanediol,
wherein the nucleotide sequence encoding an enzyme that converts 4-hydroxybutyryl-CoA into 1,4-butanediol is at least one selected from the group consisting of adh1, yiaY, adh4, adhB, mdh, eutG, fucO, dhaT, aldA, eutE, adhE1, adhE2, adh2, and polynucleotide sequences encoding the gene products thereof.

11. The expression vector according to claim 10, wherein the nucleotide sequence encoding an enzyme that converts α-ketoglutarate or succinate into 4-hydroxybutyryl-CoA is a nucleotide sequence encoding at least one of succinyl-CoA transferase, succinate semialdehyde dehydrogenase, 4-hydroxybutyrate dehydrogenase and 4-hydroxybutyryl CoA transferase.

12. The expression vector according to claim 10, wherein the nucleotide sequence encoding an enzyme that converts 4-hydroxybutyryl-CoA into 1,4-butanediol is a nucleotide sequence encoding at least one of aldehyde dehydrogenase and alcohol dehydrogenase.

13. The expression vector according to claim 11, wherein the polynucleotide comprising a nucleotide sequence for expressing succinyl-CoA transferase has at least 70% identity to SEQ ID NO. 1.

14. The expression vector according to claim 11, wherein the polynucleotide comprising a nucleotide sequence for expressing succinate semialdehyde dehydrogenase has at least 70% identity to SEQ ID NO. 2.

15. The expression vector according to claim 11, wherein the polynucleotide comprising a nucleotide sequence for expressing 4-hydroxybutyrate dehydrogenase has at least 70% identity to SEQ ID NO. 3.

16. The expression vector according to claim 11, wherein the polynucleotide comprising a nucleotide sequence for expressing 4-hydroxybutyryl-CoA transferase has at least 70% identity to SEQ ID NO. 4.

17. The expression vector according to claim 16, wherein the polynucleotide comprising a nucleotide sequence for expressing aldehyde dehydrogenase has at least 70% identity to SEQ ID NO. 5.

18. A method of producing a 1,4-butanediol, comprising:
culturing the modified microorganism according to claim 1 in a glucose-containing medium; and
recovering the 1,4-butanediol from the medium.

19. The method according to claim 18, further comprising producing polybutylene succinate (PBS) from the recovered 1,4-butanediol.

20. The method according to claim 18, further comprising producing polybutylene terephthalate (PBT) from the recovered 1,4-butanediol.

21. The modified microorganism of claim 1, wherein the modified microorganism comprises a heterologous nucleic acid encoding at least one enzyme selected from the group consisting of α-ketoglutarate dehydrogenase, α-ketoglutarate decarboxylase, succinyl-CoA transferase, succinate semialdehyde dehydrogenase, 4-hydroxybutyrate dehydrogenase, 4-hydroxybutyryl-CoA transferase, butyrate kinase, and phosphotransbutyrylase; and
a heterologous nucleic acid encoding at least one selected from the group consisting of aldehyde dehydrogenase and alcohol dehydrogenase.

22. A modified microorganism comprising the expression vector according to claim 10.
